(19) United States
(12) Patent Application Publication
(10) Pub. No.: US 2026/0137916 A1
(43) Pub. Date: May 21, 2026

(54) TREATMENT TIP, TREATMENT HANDLE AND TREATMENT DEVICE

(71) Applicant: SHENZHEN PENINSULA MEDICAL GROUP, Shenzhen (CN)

(72) Inventors: Chuancai WANG, Shenzhen (CN); Weihuo LI, Shenzhen (CN); Fei CHEN, Shenzhen (CN); Yongsheng LIANG, Shenzhen (CN)

(73) Assignee: SHENZHEN PENINSULA MEDICAL GROUP, Shenzhen (CN)

(21) Appl. No.: 19/234,047

(22) Filed: Jun. 10, 2025

Related U.S. Application Data
(63) Continuation of application No. PCT/CN2025/080778, filed on Mar. 5, 2025.

(30) Foreign Application Priority Data
Nov. 19, 2024 (CN) 202411655148.9

(51) Int. Cl. A61M 37/00

(52) U.S. Cl. CPC A61M 37/00; A61M 2037/0007

(57) ABSTRACT

Disclosed are a treatment tip, a treatment handle and a treatment device. The treatment tip includes a shell, provided with a treatment surface configured to contact a treatment area; a negative pressure assembly, a transmission assembly, a drug delivery assembly and a needle board assembly. An outlet of the negative pressure assembly is opened on the treatment surface, and is configured to apply negative pressure to the treatment area. An end of the needle board assembly is communicated with an outlet of the drug delivery assembly, and at least one hollow needle is fixed on an opposite end of the needle board assembly. The at least one hollow needle is passed through the treatment surface while the at least one hollow needle is driven by the transmission assembly. An end of the drug delivery assembly is communicated with the needle board assembly.

### CROSS-REFERENCE TO RELATED APPLICATIONS

This application is a continuation application of International Application No. PCT/CN 2025/080778, filed on Mar. 5, 2025, which claims priority to Chinese Patent Application No. 202411655148.9, filed on Nov. 19, 2024. All of the aforementioned applications are incorporated herein by reference in their entireties.

### TECHNICAL FIELD

The present application relates to the technical field of treatment devices, and in particular to a treatment tip, a treatment handle and a treatment device.

### BACKGROUND

In the related art, a liquid storage cavity is provided inside the drug skin treatment device, the drug can be a drug, a moisturizer, a lotion, or any combination thereof. When using the drug skin treatment device, the liquid drug in the liquid storage cavity can be pushed into the infusion pipe through the piston in the liquid storage cavity, then the liquid drug can be outputted to the surface of the human skin. After that, the microneedle is driven to pierce the surface of the human skin to form a pinhole, so that the liquid drug is injected into the human skin through the pinhole. In addition, the drug skin treatment device also has a negative pressure hose facing the human skin, and the negative pressure hose is used to absorb the liquid drug at the skin surface.

The drug skin treatment device usually includes a treatment tip and a treatment handle. The treatment tip is a medical device that directly acts on the human skin. In order to avoid cross infection, the treatment tip usually is a disposable consumable which needs to be replaced per one treatment for one person.

However, in existing products, the negative pressure hose is disposed at the outside of the treatment tip. When disassembling and replacing the treatment tip, the operator needs to remove the treatment tip from the treatment handle, and separate the negative pressure part at the treatment handle and the negative pressure hose outside the treatment tip, and then replace the new treatment tip. Likewise, when assembling the treatment tip, the operator needs to fix the treatment tip at the treatment handle, and insert the negative pressure part at the treatment handle into the negative pressure hose of the treatment tip. Such an operation process is cumbersome and will reduce the treatment efficiency.

### SUMMARY

The main purpose of the present application is to provide a treatment tip, a treatment handle and a treatment device, aiming to provide a treatment tip with built-in negative pressure to improve treatment efficiency.

To achieve the above purpose, the present application proposes a treatment tip, including: a shell, provided with a treatment surface configured to contact a treatment area; a negative pressure assembly, a transmission assembly, a drug delivery assembly and a needle board assembly. The negative pressure assembly, the transmission assembly, the drug delivery assembly and the needle board assembly are provided inside the shell. An outlet of the negative pressure assembly is opened on the treatment surface, and is configured to apply negative pressure to the treatment area. An end of the needle board assembly is communicated with an outlet of the drug delivery assembly, and at least one hollow needle is fixed on an opposite end of the needle board assembly; the at least one hollow needle is passed through the treatment surface while the at least one hollow needle is driven by the transmission assembly. An end of the drug delivery assembly is communicated with the needle board assembly, and is configured to inject the drug into the treatment area through the at least one hollow needle.

In an embodiment, an abutting plate is connected to an end of the shell facing the treatment surface, and the abutting plate is provided with a negative pressure hole, a transmission hole and a drug delivery hole; the negative pressure hole is capable for the negative pressure assembly to pass through the abutting plate, the transmission hole is capable for the transmission assembly to pass through the abutting plate, and the drug delivery hole is capable for the drug delivery assembly to pass through the abutting plate; the negative pressure assembly includes a filter cavity, and the filter cavity is fixed to a surface of the abutting plate of which the surface is close to the treatment surface.

In an embodiment, the filter cavity includes a first inner concave portion; the transmission hole is provided inside a projection of the first inner concave portion onto the abutting plate. The filter cavity is provided with a first filter inlet and a first filter outlet; the negative pressure assembly further includes a first negative pressure pipe and a second negative pressure pipe; the first negative pressure pipe is connected between the first filter inlet and the treatment surface; and the second negative pressure pipe is connected between the first filter outlet and the negative pressure hole.

In an embodiment, a negative pressure hollow pipe is integrally formed at a shell wall of the shell facing the first negative pressure pipe; an end of the negative pressure hollow pipe is communicated with the treatment surface, and an opposite end of the negative pressure hollow pipe is communicated with the first negative pressure pipe.

In an embodiment, the shell includes a first portion and a second portion; the first portion is provided with the abutting plate, and the second portion is provided with the treatment surface. An outer contour of the second portion gradually shrinks from a portion of the second portion contacting with the first portion toward the treatment surface; the negative pressure hollow pipe includes an inner hollow pipe and an outer hollow pipe communicated with the inner hollow pipe; the inner hollow pipe is protruded inwardly from an inner wall of the first portion and/or an inner wall of the second portion; the outer hollow pipe is protruded outwardly from an outer wall of the second portion.

In an embodiment, the first negative pressure pipe, the second negative pressure pipe and the first inner concave portion are communicated as a negative pressure loop which is projected on the abutting plate as an annular ring, and the transmission hole is disposed inside the annular ring.

In an embodiment, a distance between the first filter outlet and the abutting plate is smaller than a distance between the first filter inlet and the abutting plate.

In an embodiment, the filter cavity further includes a second inner concave portion; the drug delivery assembly includes a drug delivery pipe, and the drug delivery pipe passes through the second inner concave portion after passing through the drug delivery hole; the drug delivery assembly is provided with a drug delivery cavity; ends of the at least one hollow needles are fixed with the drug delivery cavity, and opposite ends of the at least one hollow needle are facing with the treatment surface; and the drug delivery cavity is connected to the drug delivery pipe.

In an embodiment, the drug delivery pipe is a silicone hose that is formed integrally; the drug delivery cavity includes a through hole communicated with the drug delivery pipe, and a plane where the through hole is disposed is perpendicular to the abutting plate.

In an embodiment, the drug delivery pipe is a silicone hose that is formed integrally; the drug delivery cavity includes a through hole communicated with the drug delivery pipe, and a plane where the through hole is disposed is parallel to the abutting plate.

In an embodiment, the filter cavity is disposed between the abutting plate and the drug delivery cavity; the through hole is disposed closer to a center of the shell compared with the drug delivery hole; and a gap is continuously formed between the filter cavity and the drug delivery cavity during the movement of the at least one hollow needle driven by the transmission assembly.

In an embodiment, the needle board assembly further includes a needle board, and the needle board is fixed on a side of the drug delivery assembly away from the transmission assembly; the needle board assembly further includes at least one radio frequency needle; ends of the at least one radio frequency needle are fixed on the needle board, and opposite ends of the at least one radio frequency needle are facing with the treatment surface.

In an embodiment, the needle board assembly further includes an adapter plate and an electrical connector; the adapter plate is fixed on the abutting plate; an end of the electrical connector is electrically connected to the adapter plate; an opposite end of the electrical connector is electrically connected to the at least one radio frequency needle.

In an embodiment, the transmission assembly includes a transmission hollow pillar; the transmission hollow pillar is fixed on the abutting plate, and the transmission hollow pillar passes through the annular shape after the transmission hollow pillar passes through the transmission hole; and the transmission hollow pillar is attached to a portion of the filter cavity corresponding to the first inner concave portion of the filter cavity.

In an embodiment, the transmission assembly includes a push pole and an elastic member; the push pole has an outer boss extending from an end of the push pole away from the treatment surface, and an opposite end of the push pole is fixed to the needle board assembly; and an inner boss is provided inside the transmission hollow pillar; the push pole is sleeved inside the transmission hollow pillar, and the elastic member is provided between the inner boss and the outer boss.

The present application further provides a treatment handle, detachably connected to the treatment tip as mentioned above, which includes a vacuum pump assembly, a power assembly and a drug push assembly. The vacuum pump assembly is communicated with the negative pressure assembly; the power assembly is connected to the transmission assembly, and the drug push assembly is communicated with the drug delivery assembly.

In an embodiment, a press-type snap fit is provided at the treatment handle, and the press-type snap fit is clamped with the shell.

In an embodiment, the vacuum pump assembly includes a first vacuum pump pipe and a second vacuum pump pipe, and the treatment handle further includes a filter assembly; the filter assembly includes a filter shell and a third filter; the third filter is provided inside the filter shell, and the filter shell is provided with a second filter inlet and a second filter outlet; an end of the first vacuum pump pipe is communicated with the negative pressure assembly, and an opposite end of the first vacuum pump pipe is communicated with the second filter inlet; an end of the second vacuum pump pipe is connected to the second filter outlet, and an opposite end of the second vacuum pump pipe is connected to an vacuum pump.

In an embodiment, a first position-limiting rib and a second position-limiting rib are provided inside the filter shell, and the third filter is disposed between the first position-limiting rib and the second position-limiting rib; the first position-limiting rib is disposed close to the second filter inlet, and the second position-limiting rib is close to the second filter outlet; and a length of the second position-limiting rib is greater than a length of the first position-limiting rib

The present application further provides a treatment device including: a host; the treatment tip as mentioned above and the treatment handle as mentioned above. T he treatment tip is detachably connected to the treatment handle, and the host is connected to the treatment handle.

In the technical solutions of the present application, the negative pressure assembly is provided inside the treatment tip. When the treatment tip and the treatment handle are abutted and assembled, the shell of the treatment tip is abutted against the treatment handle, and the negative pressure assembly, the transmission assembly and the drug delivery assembly in the treatment tip are synchronously abutted against the treatment handle. The treatment tip can be quickly and conveniently installed at the treatment handle with only one operation, which simplifies the installation operation and improves the treatment efficiency.

### BRIEF DESCRIPTION OF THE DRAWINGS

In order to more clearly illustrate the technical solutions in the embodiments of the present application or the related art, the following will briefly introduce the drawings that need to be used in the description of the embodiments or the related art. Obviously, the drawings in the following description are only some embodiments of this application. For those skilled in the art, without creative effort, other drawings can be obtained according to the structures shown in these drawings.

FIG. 1 is a schematic explosion structure diagram of a treatment tip according to an embodiment of the present application.

FIG. 2 is a schematic structure diagram showing positions of assemblies at an abutting plate of the treatment tip according to an embodiment of the present application.

FIG. 3 is a schematic structure diagram showing positions of assemblies at the abutting plate of the treatment tip according to another embodiment of the present application.

FIG. 4 is a schematic structure diagram showing positions of assemblies at the abutting plate of the treatment tip according to yet another embodiment of the present application.

FIG. 5 is a schematic structure diagram of a transmission assembly of the treatment tip according to an embodiment of the present application.

FIG. 6 is a schematic structure diagram of a drug delivery assembly of the treatment tip according to an embodiment of the present application.

FIG. 7 is a schematic structure diagram of a needle board assembly of the treatment tip according to an embodiment of the present application.

FIG. 8 is a schematic structure diagram of a negative pressure assembly of the treatment tip according to an embodiment of the present application.

FIG. 9 is a schematic structure diagram of a filter cavity of the treatment tip according to an embodiment of the present application.

FIG. 10 is a schematic structure diagram of a first negative pressure pipe and a second negative pressure pipe of the treatment tip according to an embodiment of the present application.

FIG. 11 is a schematic top view structure diagram of the negative pressure assembly installed in the shell of the treatment tip according to an embodiment of the present application.

FIG. 12 is a schematic left view structure diagram of the negative pressure assembly installed in the shell of the treatment tip according to an embodiment of the present application.

FIG. 13 is a schematic structure diagram of the treatment handle according to an embodiment of the present application.

FIG. 14 is a schematic structure diagram of the filter assembly of the treatment handle according to an embodiment of the present application.

FIG. 15 is a schematic structure diagram of the drug delivery assembly of the treatment handle according to an embodiment of the present application.

FIG. 16 is a schematic partial structural diagram of a cross-sectional view of a shell and a negative pressure assembly according to an embodiment of the present application.

The realization of the objective, functional characteristics, and advantages of the present application are further described with reference to the accompanying drawings.

### DETAILED DESCRIPTION OF THE EMBODIMENTS

The technical solutions of the embodiments of the present application will be described in detail below with reference to the accompanying drawings. It is obvious that the embodiments described are only some rather than all of the embodiments of the present application. All other embodiments obtained by those skilled in the art based on the embodiments of the present application without creative efforts shall fall within the claimed scope of the present application.

It should be noted that all the directional indications (such as up, down, left, right, front, rear . . . ) in the embodiments of the present application are only used to explain the relative positional relationship, movement, or the like of the parts in a certain posture. If the specific posture changes, the directional indication will change accordingly.

Besides, the descriptions associated with, e.g., “first” and “second,” in the present application are merely for descriptive purposes, and cannot be understood as indicating or suggesting relative importance or impliedly indicating the number of the indicated technical feature. Therefore, the feature associated with “first” or “second” can expressly or impliedly include at least one such feature. Further, if “and/or” appears throughout the text, it includes three parallel schemes. Taking “A and/or B” as an example, it includes the scheme A, or the scheme B, or the scheme that the scheme A and the scheme B satisfy synchronously. In addition, the technical solutions of the various embodiments can be combined with each other, but the combinations must be based on the realization of those skilled in the art. When the combination of technical solutions is contradictory or cannot be achieved, it should be considered that such a combination of technical solutions does not exist, nor does it fall within the scope of the present application.

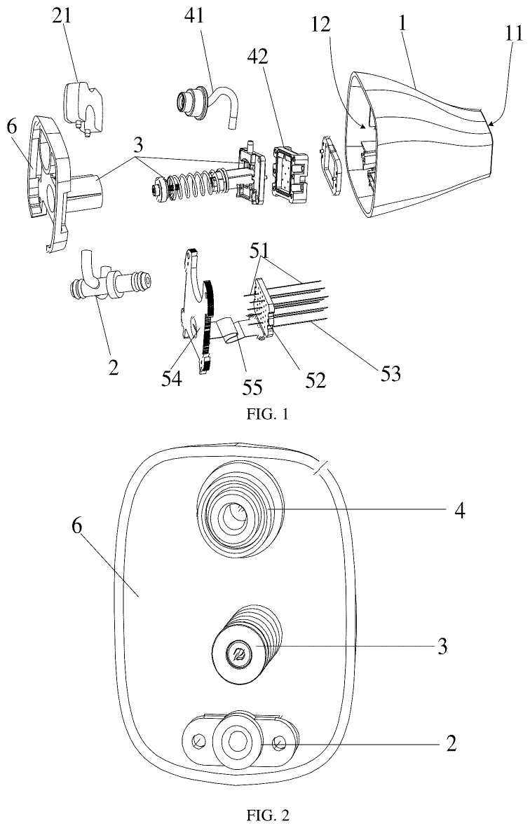

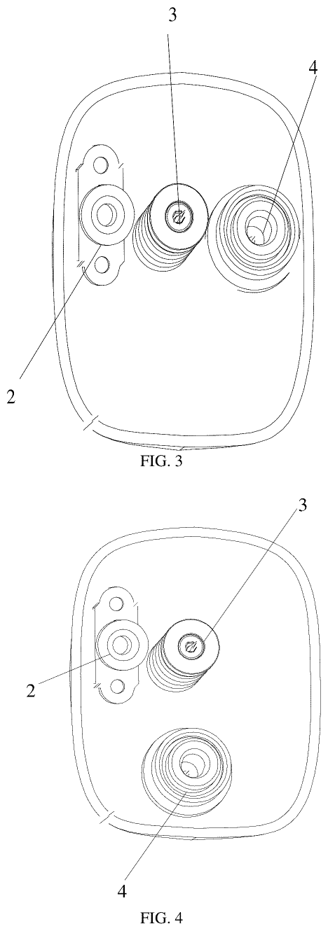

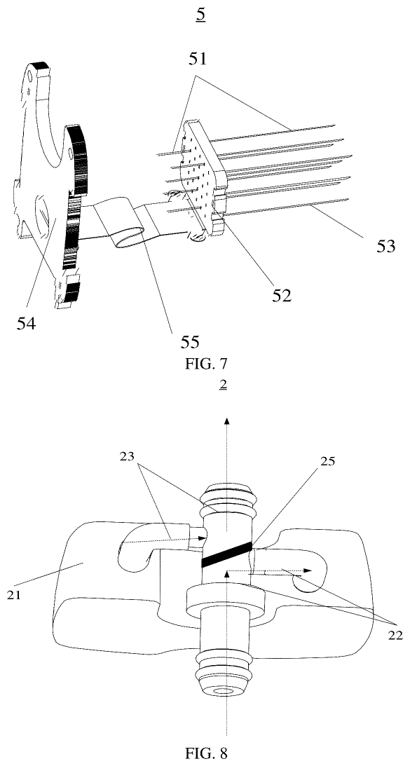

As shown in FIG. 1 to FIG. 16, the present application proposes a treatment tip including a shell 1. The shell 1 is provided with a treatment surface 11. The treatment surface 11 is configured to contact a treatment area. A negative pressure assembly 2, a transmission assembly 3, a drug delivery assembly 4 and a needle board assembly 5 are provided in the installation cavity. An outlet of the negative pressure assembly 2 is opened on the treatment surface 11, and is configured to apply negative pressure to the treatment area. One end of the needle board assembly 5 is communicated with the outlet of the drug delivery assembly 4, and at least one hollow needle 51 is fixed on an opposite end of the needle board assembly 5. The at least one hollow needle 51 is passed through the treatment surface 11 while the at least one hollow needle 51 is driven by the transmission assembly 3. One end of the drug delivery assembly 4 is communicated with the needle board assembly 5, so as to introduce drug into the treatment area through the hollow needle 51.

In this embodiment, the shell 1 is a part provided with an installation cavity, which is empty inside and can hold other parts of the treatment tip. The shell 1 can be made of plastic, ceramic, and the like. For cost, weight and other considerations, the shell 1 is generally made of plastic. The peripheral wall of the shell 1 is in an annular shape, to form an installation cavity. The installation cavity has two opposite surfaces, namely, the treatment surface 11 and the connection surface 12. The treatment surface 11 is configured to contact the treatment area. The treatment area can be the human skin, such as face, arm, and the like. The treatment tip needs to contact the skin, from a medical point of view, in order to avoid cross infection, a treatment tip is usually used for treating only one person, so that the treatment tip usually belongs to consumables. The treatment tip is detachably connected to the treatment handle. The treatment drug or treatment energy in the treatment handle passes through the treatment tip and reaches the treatment area for achieving treatment. The drug can be a medicine, a moisturizer, a lotion, or any combination thereof. When treatment is finished, the treatment tip is removed from the treatment handle and replaced with an unused treatment tip, and then another person can be treated. The surface of the treatment tip connected to the treatment handle is the connection surface 12. Usually, the treatment surface 11 and the connection surface 12 are the cross-sections at two opposite ends of the treatment tip, but for some abnormal or specific treatment tips, the treatment surface 11 and the connection surface 12 are two adjacent cross-sections of the treatment tip.

The negative pressure assembly 2, the transmission assembly 3, the drug delivery assembly 4 and the needle board assembly 5 are provided in the installation cavity. Two ends of the negative pressure assembly 2 are respectively provided at the treatment surface 11 and the connection surface 12. The end of the negative pressure assembly 2 provided at the connection surface 12 is connected to the negative pressure pump at the treatment handle, so that the negative pressure pump at the treatment handle generates negative pressure and acts on the treatment area through the negative pressure assembly 2. One end of the transmission assembly 3 is provided at the connection surface 12, and is connected to the power assembly at the treatment handle. After the power assembly moves, the force is transmitted to the transmission assembly 3. an opposite end of the transmission assembly 3 is connected to the needle board assembly 5, so that the transmission assembly 3 pushes the needle board assembly 5 to move toward the treatment area, and then the hollow needle 51 of the needle board assembly 5 penetrates into the treatment area. In addition, one end of the drug delivery assembly 4 is provided at the connection surface 12, and is communicated with with the liquid outlet assembly of the treatment handle. an opposite end of the drug delivery assembly 4 is connected to the needle board assembly 5, so that the drug inside the treatment handle is introduced from the liquid outlet assembly, and then flows into the hollow needle 51 of the needle board assembly 5 through the drug delivery assembly 4. After that, as the hollow needle 51 penetrates into the treatment area, the drug is introduced into the treatment area. The drug can be in various forms, such as liquid, gas, powder, paste, or a combination of multiple forms, and can be introduced from the drug delivery assembly 4 into the hollow needle 51.

In this embodiment, the negative pressure assembly 2, the transmission assembly 3, the drug delivery assembly 4 and the needle board assembly 5, which are used for treatment, are integrated into the shell 1. When the treatment tip and the treatment handle are abutted and assembled, the shell 1 of the treatment tip is abutted against the treatment handle, and the negative pressure assembly 2, the transmission assembly 3 and the drug delivery assembly 4 in the treatment tip are synchronously abutted against the treatment handle. The treatment tip can be quickly and conveniently installed at the treatment handle with only one operation, which simplifies the installation operation and improves the treatment efficiency.

In an embodiment, an abutting plate 6 is connected to one end of the shell 1 opposite to the treatment surface 11. The abutting plate 6 is provided with a negative pressure hole, a transmission hole and a drug delivery hole. The negative pressure hole is capable for the negative pressure assembly 2 to pass through the abutting plate 6. The transmission hole is capable for the transmission assembly 3 to pass through the abutting plate 6, and the drug delivery hole is capable for the drug delivery assembly 4 to pass through the abutting plate 6.

In this embodiment, an abutting plate 6 is connected to one end of the shell 1 opposite to the treatment surface 11, and it can be understood that the abutting plate 6 is provided at the connection surface 12 of the shell 1. The shape of the abutting plate 6 is the same as or similar to the shape of the connection surface 12, so that the abutting plate 6 can cover the surface of the shell 1. In some embodiments, the periphery of the abutting plate 6 covers the periphery of the connection surface 12. That is, the abutting plate 6 has a plane and a circle of sidewalls protruding from the plane toward the treatment surface 11. In some embodiments, the abutting plate 6 is provided inside the installation cavity, and the periphery of the abutting plate 6 is tightly attached to the inner wall of the shell 1. Further, the abutting plate 6 has a plane and a circle of sidewalls protruding from the plane toward the connection surface 12. In some embodiments, the abutting plate 6 is a flat board, and the peripheral edge of the abutting plate 6 completely or approximately matches with the connection surface 12 at the shell 1. Regardless of how the abutting plate 6 is arranged, a negative pressure hole, a transmission hole, and a drug delivery hole are provided at a position of the abutting plate 6 corresponding to the connection surface 12, which are respectively used for the negative pressure assembly 2, the transmission assembly 3, and the drug delivery assembly 4 to pass through, so that the negative pressure assembly 2, the transmission assembly 3, and the drug delivery assembly 4 can communicate with the treatment tip and the treatment handle. Besides, the activity space is limited inside the treatment tip, to avoid the negative pressure assembly 2, the transmission assembly 3, and the drug delivery assembly 4 from being entangled with each other when the treatment tip and the treatment handle are moved during the treatment process.

In an embodiment, the drug delivery hole and the negative pressure hole are disposed around the transmission hole.

In this embodiment, the drug delivery hole and the negative pressure hole are disposed around the transmission hole. That is, the drug delivery assembly 4 and the negative pressure assembly 2 are disposed around the transmission assembly 3. For example, the drug delivery hole, the transmission hole, and the negative pressure hole are all provided at the abutting plate 6, and centers of these three holes are located at a straight line or a bent curve. The shape of the abutting plate 6 is similar to the shape of the connection surface 12 of the shell 1, which can be in the rectangular shapes or approximately rectangular shapes. The transmission hole is provided at the center or close to the center of the abutting plate 6. The drug delivery hole and the negative pressure hole are provided at the edge position of the abutting plate 6, that is, the position away from the center. When transmitting power to the needle board assembly 5, the transmission assembly 3 will also move. The position that the transmission assembly 3 moves to is the center of the abutting plate 6 or close to the center of the abutting plate 6, rather than at the edge of the abutting plate 6, so that the transmitted power can produce less vibration, thereby minimizing the impact on the vibration of the treatment tip and improving the stability of the treatment tip. In addition, the drug delivery hole is set at the edge of the abutting plate 6. Correspondingly, the drug push assembly of the treatment handle can be set around the treatment handle. The drug push assembly has a drug storage box, which can facilitate removal or injection of drug.

In other embodiments, the drug delivery hole and the transmission hole are set around the negative pressure hole.

In this embodiment, the drug delivery hole and the transmission hole are set around the negative pressure hole, that is, the negative pressure hole is set at the center of the abutting plate 6 or is set close to the center of the abutting plate 6. Then the negative pressure assembly 2 can extend multiple negative pressure channels from the center of the abutting plate 6 to the edge of the shell 1. The adsorption ports of the multiple negative pressure channels are symmetrically distributed at the treatment surface 11, so that the treatment area can be adsorbed more evenly and efficiently from multiple angles simultaneously, and impurities can be better adsorbed from multiple positions. In this way, more impurities are adsorbed away, thereby improving the treatment efficiency.

In other embodiments, the transmission hole and the negative pressure hole are disposed around the drug delivery hole.

In this embodiment, the drug delivery hole is provided at the center of the abutting plate 6, or is provided close to the center of the abutting plate 6. The transmission hole and the negative pressure hole are disposed at the edge of the abutting plate 6 around the drug delivery hole. Since the drug delivery assembly 4 needs to deliver drug to the hollow needle 51 below the drug delivery assembly 4, the drug delivery hole is provided at the center or close to the center of the abutting plate 6. In this way, the route from the drug delivery assembly 4 to the hollow needle 51 is a vertical downward route. During the treatment process, when the operator holds the treatment handle, the drug delivery assembly 4 passes through the drug delivery hole in the center of the abutting plate 6, and then delivers the drug to the hollow needle 51 directly below the drug delivery assembly 4, so that drug can enter the hollow needle 51 more efficiently under the action of gravity. In addition, the transmission hole is set at the edge of the abutting plate 6 near the position of the thumb of the operator's hand holding the treatment handle, correspondingly, the power assembly at the treatment handle, such as the motor, is also provided close to the thumb of the operator's hand, so that the heavier motor in the treatment handle is closest to the force point for the human hand to hold, and the treatment handle can be operated more flexibly.

In different embodiments, according to the different structural features of the negative pressure assembly 2, the transmission assembly 3, and the drug delivery assembly 4, and according to the structural features of each corresponding part at the treatment handle, positions of the drug delivery hole, the negative pressure hole, and the transmission hole can also be set more flexibly.

In an embodiment, a positioning structure 14 protrudes from the inner wall of the shell 1, and the positioning structure 14 is abutted against the surface of the abutting plate 6 of which the surface is close to the treatment surface 11.

In this embodiment, the shell 1 is made of plastic, such as polyethylene (PE), and the positioning structure 14 is formed by injection molding. The positioning structure 14 protrudes from the inner wall of the shell 1 and is abutted against the abutting plate 6, so that the abutting plate 6 is fixed by the positioning structure 14 and the treatment tip. In some embodiments, there can be multiple positioning structures 14, and multiple positioning structures 14 abut against multiple lower edges of the abutting plate 6, which has a better fixing effect. In some embodiments, the positioning structure 14 can be shaped in a block. In some embodiments, the positioning structure 14 is in an annular shape, which can abut against the periphery of the abutting plate 6. In some embodiments, the positioning structure 14 has an internal thread groove. Correspondingly, the abutting plate 6 has the avoidance hole, which is convenient for the screw to pass through the abutting plate 6 and then be screwed into the thread groove of the positioning structure 14, to fix the abutting plate 6 in the shell 1.

In an embodiment, the negative pressure assembly 2 includes a filter cavity 21, and the filter cavity 21 is fixed to a surface of the abutting plate 6 of which the surface is close to the treatment surface 11.

In this embodiment, the filter cavity 21 is a space with a cavity, and a filter piece is provided inside the cavity and used for filtering. During the treatment process, the treatment surface 11 of the treatment tip attaches tightly to the treatment area, and the negative pressure assembly 2 is used to absorb the treatment area, so that the hollow needle 51 can penetrate into the treatment area, and then the drug delivery assembly 4 introduces the drug into the treatment area through the hollow needle 51. Since the treatment surface 11 attaches tightly to the treatment area, the negative pressure assembly 2 will absorb some impurities in the treatment area during absorption. In addition, when withdrawing from the treatment area, the hollow needle 51 will inevitably bring out some drug or impurities at the treatment area. For example, if the treatment area is skin, there may be impurities, such as hair, sweat, and skin debris, at the surface of the skin. When withdrawing from the treatment area, the hollow needle 51 will bring out impurities, such as blood and dermal tissue in the dermis. Therefore, the negative pressure assembly 2 is provided with a filter cavity 21, in which the above impurities can be adsorbed, to prevent the impurities from entering the negative pressure pump of the treatment handle along with the negative pressure assembly 2. The filter cavity 21 is provided inside the shell 1, and is provided at the surface of the abutting plate 6 facing the treatment surface 11, which actually means that the filter cavity 21 is provided close to the treatment surface 11. In this way, the distance from the treatment area to the filter cavity 21 can also be shorten, so that impurities are adsorbed as soon as possible. The negative pressure assembly 2 has a negative pressure channel from the treatment surface 11 to the treatment handle, and the filter cavity 21 is provided as close to the treatment surface 11 as possible, so that the impurities are quickly adsorbed at the front end of the negative pressure channel, thereby preventing impurities flowing into the rear end of the negative pressure channel.

In an embodiment, a projection of the filter cavity 21 onto the abutting plate 6 includes a first inner concave portion 211; the transmission hole is provided inside the first inner concave portion 211.

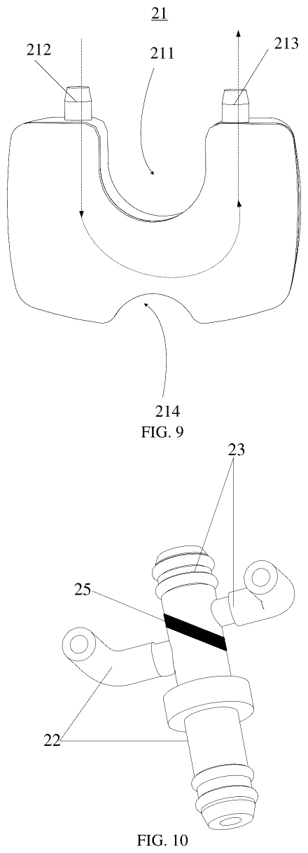

In this embodiment, the filter cavity 21 is a special-shaped structure. Specifically, with reference to FIG. 9, a first inner concave portion 211 is provided on the projection of the abutting plate 6, which is in a “concave” shape. Correspondingly, the negative pressure channel is in a “U-shape” in the filter cavity 21. In this case, impurities flows from the upper left part of the “concave” shape, and flows out from the upper right part of the “concave” shape. In addition, the first inner concave portion 211 in the “concave” shape further plays the avoidance role. The transmission hole opened on the abutting plate 6 is provided inside a projection of the first inner concave portion 211 onto the abutting plate 6, so that the transmission assembly 3 passes through the transmission hole and is provided close to the first inner concave portion 211, and then acts on the needle board assembly 5 below the filter cavity 21.

In an embodiment, the filter cavity 21 is provided with a first filter inlet 212 and a first filter outlet 213. The negative pressure assembly 2 further includes a first negative pressure pipe 22 and a second negative pressure pipe 23. The first negative pressure pipe 22 is connected between the first filter inlet 212 and the treatment surface 11. The second negative pressure pipe 23 is connected between the first filter outlet 213 and the negative pressure hole.

In this embodiment, the negative pressure assembly 2 has a negative pressure channel. Specifically, as shown in FIG. 8, FIG. 9 and FIG. 12, the dotted line represents the negative pressure channel, and the negative pressure channel is composed of a first negative pressure pipe 22, a filter cavity 21 and a second negative pressure pipe 23. The airflow enters the first negative pressure pipe 22 from the treatment surface 11, and then enters the filter cavity 21. After flowing out from the filter cavity 21, the airflow enters the second negative pressure pipe 23. One end of the second negative pressure pipe 23 is fixed in the negative pressure hole, and an opposite end of the second negative pressure pipe 23 is communicated with the filter cavity 21. Correspondingly, the filter cavity 21 has a first filter inlet 212 and a first filter outlet 213. One end of the first negative pressure pipe 22 faces the treatment surface 11 and is configured to adsorb the treatment area. an opposite end of the first negative pressure pipe 22 communicates with the first filter inlet 212. One end of the second negative pressure pipe 23 communicates with the first filter outlet 213. an opposite end of the second negative pressure pipe 23 is fixed in the negative pressure hole and communicates with the negative pressure pump of the treatment handle. Therefore, the negative pressure channel passes through the treatment tip, and passes from the treatment surface 11 to the negative pressure pump of the treatment handle.

In an embodiment, a negative pressure hollow pipe 13 is integrally formed at a position of the shell wall of the shell 1 facing the first negative pressure pipe 22. One end of the negative pressure hollow pipe 13 communicates with the treatment surface 11, and an opposite end of the negative pressure hollow pipe 13 communicates with the first negative pressure pipe 22.

In this embodiment, the first negative pressure pipe 22 communicates the treatment surface 11 with the filter cavity 21. The first negative pressure pipe 22 can be provided close to the inner wall of the shell 1, or can be attached tightly to the inner wall of the shell 1. Or, the first negative pressure pipe 22 can be integrally formed with the negative pressure hollow pipe 13 of the shell 1. In some embodiments, the first negative pressure pipe 22 includes a rubber pipe and the negative pressure hollow pipe 13. The negative pressure hollow pipe 13 is a pipe protruding from the inner wall of the shell 1, and is integrally formed. One end of the negative pressure hollow pipe 13 faces the treatment surface 11, and an opposite end of the negative pressure hollow pipe 13 communicates with the rubber pipe. The rubber pipe can be inserted into the negative pressure hollow pipe 13 for communication, or the negative pressure hollow pipe 13 can be inserted into the rubber pipe for communication. an opposite end of the rubber pipe communicates with the first filter inlet 212. In an embodiment, a first filter is provided in the negative pressure hollow pipe 13, which is configured to filter impurities in the adsorbed air. The first filter can be the sponge. Sponge is a porous material that facilitates gas circulation to form a good negative pressure channel, and further has good water absorption.

In an embodiment, the shell 1 further includes a connection surface 12 provided opposite to the treatment surface 11, and the connection surface 12 is abutted against the treatment handle. The acreage of the connection surface 12 is larger than the acreage of the treatment surface 11.

In this embodiment, the connection surface 12 is configured to abut against the treatment handle, and multiple parts need to be provided at the connection surface 12. The connection surface 12 further needs to match and abut against the treatment handle, so that the acreage of the connection surface 12 should be larger. The treatment surface 11 is configured to contact the treatment area, and the doctor or operator needs to pay attention to the treatment area. Therefore, the acreage of the treatment surface 11 is set to be smaller, which can be equal to the acreage of the direct treatment part, such as the hollow needle 51. In this way, the acreage of the treatment surface 11 is relatively smaller.

In an embodiment, the shell 1 includes a first portion 15 and a second portion 16. The first portion 15 includes a connection surface 12, and the connection surface 12 is provided with an abutting plate 6. The second portion 16 includes the treatment surface 11. The outer contour of the second portion 16 gradually shrinks from the portion of the second portion 16 contacting with the first portion 15 to the treatment surface 11.

The first portion 15 and the second portion 16 are both parts of the shell 1, and they are distinguished only in the outer contour. In this case, it does not mean that there is a physical division between the first portion 15 and the second portion 16. The first portion 15 may be in a column, a square shape, or other cylindrical shapes, and the like. A connection surface 12 is provided at the first portion 15, which may abut against a treatment handle. A structure extends from a surface of the first portion 15 away from the connection surface 12, and gradually shrinks at the peripheral wall of the structure, until the structure shrinks to the treatment surface 11, thereby forming the second portion 16. The shrinkage may be continuous or discontinuous, or may combine continuous shrinkage and discontinuous shrinkage.

In an embodiment, the negative pressure hollow pipe 13 includes an inner hollow pipe 131 and an outer hollow pipe 132 communicating with the inner hollow pipe 131. The inner hollow pipe 131 is protruding inwardly and provided at the inner wall of the first portion 15 and/or the inner wall of the second portion 16. The outer hollow pipe 132 is protruding outwardly and provided at the outer wall of the second portion 16.

As shown in FIG. 16, the negative pressure hollow pipe 13 is formed at the shell wall of the shell 1. Part of the negative pressure hollow pipe 13 is provided inside the shell 1, that is, the inner hollow pipe 131 is provided inside the shell 1. The other part of the negative pressure hollow pipe 13 is provided outside the shell 1, that is, the outer hollow pipe 132 is provided outside the shell 1. The inner hollow pipe 131 is provided at the inner wall of the first portion 15. Or the inner hollow pipe 131 is partially provided at the inner wall of the first portion 15 and partially provided at the inner wall of the second portion 16. Or the inner hollow pipe 131 is completely provided at the inner wall of the second portion 16. The outer hollow pipe 132 is provided at the outer wall of the second portion 16. In this way, not only the length direction of the entire negative pressure hollow pipe 13 is perpendicular to the treatment surface 11, but also the entire negative pressure hollow pipe 13 further penetrates the shell 1 and communicates the outside of the shell 1 with the inside of the shell 1. As shown in FIG. 16, there is a thickened line at the left side of the inner hollow pipe 131 and there is also a thickened line at the right side of the outer hollow pipe 132. Both these two lines belong to a part of the shell 1. Thickening is used to explain the distinction between inward protrusion and outward protrusion of the first portion 15 and the second portion 16, or thickening is used to explain the distinction between the inner wall and the outer wall of the first portion 15 and the second portion 16, which does not mean that the thickness of this portion of the shell is thickened.

In an embodiment, the first negative pressure pipe 22, the second negative pressure pipe 23 and the first inner concave portion 211 are communicated as a negative pressure loop which is projected on the abutting plate 6 as an annular ring 24, and the transmission hole is disposed inside the annular ring 24.

In this embodiment, the first negative pressure pipe 22 and the second negative pressure pipe 23 are rubber pipes. The first negative pressure pipe 22 includes a first vertical section 221 and a first horizontal section 222, and the second negative pressure pipe 23 includes a second vertical section 231 and a second horizontal section 232. The first vertical section 221 and the second vertical section 231 are coaxial. The negative pressure channel which is formed by the first negative pressure pipe 22, the “concave” filter cavity 21, and the second negative pressure pipe 23, is projected on the abutting plate 6 in the annular shape 24. The transmission hole is located in the annular shape 24. The transmission assembly 3 passes through the annular shape 24 and acts on the needle board assembly 5.

In an embodiment, the first negative pressure pipe 22 is fixed to the second negative pressure pipe 23 to form an integrated structure through a connector 25.

The connector 25 connects one pipe wall of the first negative pressure pipe 22 with one pipe wall of the second negative pressure pipe 23, to form the first negative pressure pipe 22 and the second negative pressure pipe 23 to an integral structure. In addition, there is no communication between the first negative pressure pipe 22 and the second negative pressure pipe 23, and only by a filter cavity 21, the first negative pressure pipe 22 can be communicated with the second negative pressure pipe 23. Or, a rubber pipe can be set at the connection position between the first negative pressure pipe 22 and the second negative pressure pipe 23, and the connector 25 with the solid construction is set at the rubber pipe to separate the first negative pressure pipe 22 and the second negative pressure pipe 23.

In this embodiment, both the first negative pressure pipe 22 and the second negative pressure pipe 23 are rubber pieces. The first negative pressure pipe 22 is connected to the second negative pressure pipe 23 through the connector 25 to form an integral structure. The connector 25 is a solid structure, which will not communicate the first negative pressure pipe 22 with the second negative pressure pipe 23. For example, there is a rubber block at the intersection of the first vertical section 221 and the first horizontal section 222 and the intersection of the second vertical section 231 and the second horizontal section 232. The rubber block is the connector 25, which can be used to allow the first negative pressure pipe 22 and the second negative pressure pipe 23 to form an integrated structure. In this way, it is convenient for processing and assembling the treatment tip. When assembling, the operator picks up the integrated structure, inserts the first vertical section 221 into the negative pressure hollow pipe 13, fixes the second vertical section 231 in the negative pressure hole, and then communicates the first horizontal section 222 with the first filter inlet 212 of the filter cavity 21 and communicates the second horizontal section 232 with the first filter outlet 213 of the filter cavity 21 respectively. The first negative pressure pipe 22 and the second negative pressure pipe 23 are both made of rubber material and have certain elasticity. The filter cavity 21 can be made of PE with certain rigidity. The first filter inlet 212 and the first filter outlet 213 also have certain rigidity. The rigid material PE can be inserted into the rubber pipe with certain elasticity for installation. The installation is quick and convenient. In an embodiment, the first negative pressure pipe 22, the second negative pressure pipe 23 and the connector 25 are all made of rubber and are formed by injection molding.

As shown in FIG. 8 and FIG. 10, the first negative pressure pipe 22 includes a first vertical section 221 and a first horizontal section 222, and the second negative pressure pipe 23 includes a second vertical section 231 and a second horizontal section 232. The first vertical section 221 and the second vertical section 231 are provided at a longer hollow hose, with a connector 25 provided at the middle of the hollow hose. The connector is a rubber block with a solid structure fixed on the middle of the longer hollow hose, so that the longer hollow hose is separated from the upper and lower parts of the longer hollow hose, and the upper part of the longer hollow hose cannot communicate with the lower part of the longer hollow hose. In addition, the first horizontal section 222 communicates with the longer hollow hose below the connector, and the second horizontal section 232 communicates with the longer hollow hose above the connector. Thus, the second vertical section 231 is disposed above the longer hollow hose and the upper part of the longer hollow hose communicates with the second horizontal section 232. The first vertical section 221 is disposed below the longer hollow hose, and the lower part of the longer hollow hose communicates with the first horizontal section 222. The first negative pressure pipe 22, the second negative pressure pipe 23 and the connector 25 form an integrated structure, which can be integrally formed by injection molding. The integrated structure is a structure approximately in the shape of a cross.

In an embodiment, the distance between the first filter outlet 213 and the abutting plate 6 is less than the distance between the first filter inlet 212 and the abutting plate 6.

In this embodiment, the first filter outlet 213 is closer to the abutting plate 6 with reference to the first filter inlet 212. In this way, the negative pressure channel is an upward channel in the filter cavity 21. Besides, it is difficult to adsorb impurities due to the influence of gravity, and impurities will remain in the filter cavity 21. In addition, the first filter outlet 213 and the second filter inlet 212 are provided at the same side of the filter cavity 21, so that the flow channel of the negative pressure channel in the filter cavity 21 is approximately in the “U” shape, and the airflow can flow in the filter cavity 21 as much as possible, so that more impurities or liquids can be adsorbed. Arrows of the dotted line segments in FIG. 8, FIG. 9, and FIG. 12 represents the airflow movement route of the negative pressure.

In an embodiment, a second filter is provided in the filter cavity 21.

In this embodiment, the second filter is provided in the filter cavity 21, which can further adsorb impurities and make impurities remain in the filter cavity 21. The second filter can be the sponge.

In an embodiment, the filter cavity 21 further includes a second inner concave portion 214. The drug delivery assembly 4 includes a drug delivery pipe 41. The drug delivery pipe 41 passes through the second inner concave portion 214 after passing through the drug delivery hole.

In this embodiment, the filter cavity 21 further has a second inner concave portion 214. The second inner concave portion 214 is opposite to the first inner concave portion 211 or is adjacent to the first inner concave portion 211. According to the relative arrangement of the drug delivery hole, the drug delivery pipe 41 passes through the second inner concave portion 214. The size/diameter of the drug delivery pipe 41 is smaller than the size/diameter of the transmission assembly 3. Correspondingly, the shape of the second inner concave portion 214 is smaller than the shape of the first inner concave portion 211.

In an embodiment, the drug delivery assembly 4 includes a drug delivery cavity 42. One end of the hollow needle 51 is fixed in the drug delivery cavity 42, and an opposite end of the hollow needle 51 faces the treatment surface 11. The drug delivery cavity 42 is connected to the drug delivery pipe 41.

In this embodiment, the hollow needle 51 communicates with the drug delivery cavity 42. Specifically, one end of the hollow needle 51 is inserted into the drug delivery cavity 42 to achieve communication, and the outer periphery of the hollow needle 51 is sealed at the part connected with the drug delivery cavity 42. The drug delivery cavity 42 further has a drug inlet, which communicates with the drug delivery pipe 41. The drug is discharged from the treatment handle and enters the drug delivery cavity 42 through the drug delivery pipe 41. When the needle board assembly 5 moves toward the treatment surface 11, the drug push assembly of the treatment handle works, so that the drug enters the hollow needle 51 and enters the inside of the treatment area along the hollow needle 51. The drug delivery cavity 42 is provided below the filter cavity 21, and when driven by the transmission force of the transmission assembly 3, the drug delivery cavity 42 moves toward the treatment surface 11.

In an embodiment, the drug delivery pipe 41 is a silicone hose that is formed integrally.

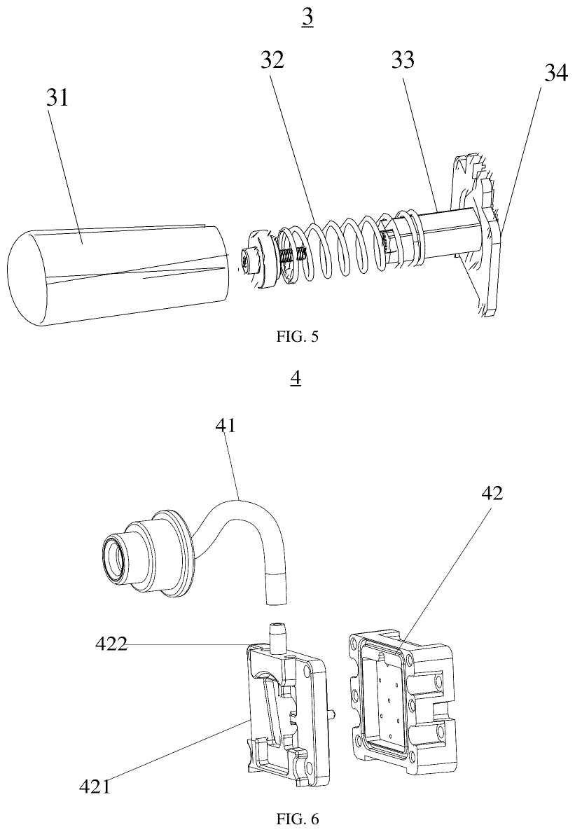

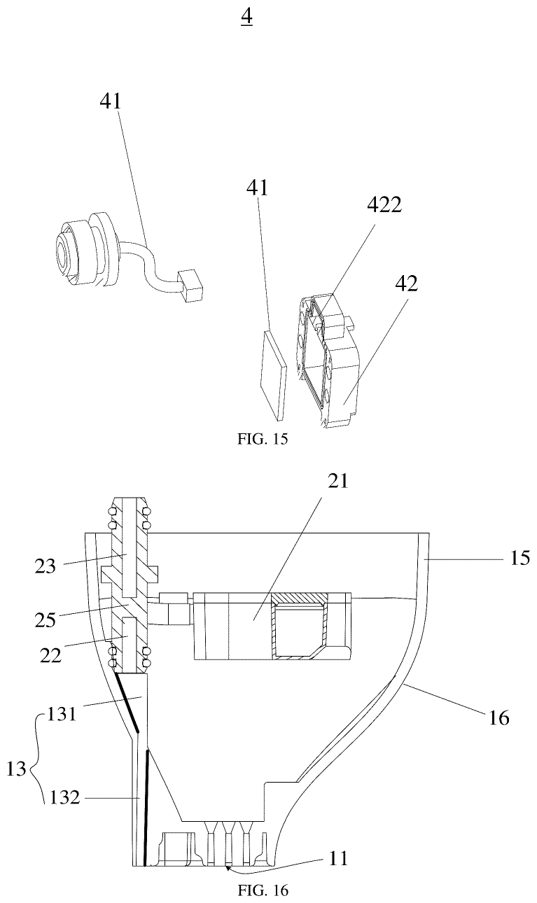

In this embodiment, the drug delivery pipe 41 is a silicone hose, which communicates with the drug delivery cavity 42. When the hollow needle 51 moves, one end of the hollow needle 51 is fixed in the drug delivery cavity 42, so that the drug delivery cavity 42 will also move synchronously, and will inevitably drive the drug delivery pipe 41 connected to the drug delivery cavity 42 to move. The drug delivery pipe 41 is a silicone hose, so that when the drug delivery cavity 42 moves, the drug delivery pipe 41 is deformed and communicates with the drug delivery cavity 42. Combined with the second inner concave portion 214, the drug delivery pipe 41 is in the L-shape, S-shape, U-shape, V-shape, wavy-shape, and the like, so as to leave activity space for the drug delivery pipe 41 to fold during movement. It should be noted that the silicone hose is elastic and easily deformed when subjected to force. The above-mentioned shape, such as L-shape, S-shape, U-shape, V-shape, wavy-shape, and the like, refer to the shape of the drug delivery pipe 41 when the drug delivery pipe 41 is not subjected to external force and not connected to other parts. As shown in FIG. 6 and FIG. 15, the silicone hose (namely, the drug delivery pipe 41) is not connected to other parts and is approximately in the L-shape. This facilitates flexible expansion and contraction or forming a specified bending path when the drug delivery pipe 41 moves synchronously with the drug delivery cavity 42, and the state is more stable. Of course, the process of producing and processing the silicone hose with a curved shape will be more troublesome, and some will require more complicated molds. Therefore, the silicone hose can be shaped in a straight line when not subjected to external force. One end of the straight silicone hose communicates with the drug delivery hole, and an opposite end of the straight silicone hose communicates with the through hole 422 of the drug delivery cavity 42. After the silicone hose is installed, since the length of the silicone hose is greater than the distance between the drug delivery hole and the through hole 422, the silicone hose will also bend into the shape shown in FIG. 6, which will not affect the drug flowing from the silicone hose to the drug delivery cavity 42 when the drug delivery cavity 42 is moving.

In an embodiment, the filter cavity 21 is provided between the abutting plate 6 and the drug delivery cavity 42. One end of the silicone hose is provided in the drug delivery hole, and an opposite end of the silicone hose communicates with the drug delivery cavity 42.

In this embodiment, one end of the silicone hose is provided in the drug delivery hole, and an opposite end of the silicone hose is provided at the drug delivery cavity 42. There is a filter cavity 21 between the silicone hose and the drug delivery cavity 42. In this way, the activity space of the silicone hose along the length direction at least includes the space along the thickness direction of the filter cavity 21, so that drug can also flow into the inner portions when the silicone hose moves.

In an embodiment, the drug delivery cavity 42 includes a through hole 422 communicated with the drug delivery pipe 41, and the plane on which the through hole 422 is disposed is perpendicular to the abutting plate 6.

As shown in FIG. 6, in this embodiment, the drug delivery cavity 42 has a through hole 422, which protrudes from the peripheral sidewall of the drug delivery cavity 42. The plane where the through hole 422 is located is perpendicular to the abutting plate 6, so that the drug delivery pipe 41 made of rubber can be conveniently inserted into the through hole 422. In addition, by setting the second inner concave portion 214 of the filter cavity 21 above the drug delivery cavity 42, it is convenient for the drug delivery pipe 41 to tilt downward and a certain installation space will be reserved, so that the drug delivery pipe 41 can be inserted into the through hole 422 horizontally. There is a certain distance between the projection of the through hole 422 on the abutting plate 6 and the drug delivery hole. The drug delivery cavity 42 is further provided with a drug delivery cavity cover 421, so that a closed cavity can be formed in the drug delivery cavity 42. In an embodiment, the above-mentioned through hole 422 is provided at the sidewall of the drug delivery cavity cover 421. In an embodiment, the drug delivery pipe 41 is a silicone hose, which is straight when there is no external force. When the drug delivery pipe 41 is installed, the drug delivery pipe 41 is bent, so that one end of the drug delivery pipe 41 communicates with the through hole 422, to make the drug delivery pipe 41 bent into the L-shape or an arc shape.

As shown in FIG. 15, in an embodiment, the drug delivery cavity 42 includes a through hole 422, and the through hole 422 protrudes from the peripheral sidewall of the drug delivery cavity 42. The plane where the above-mentioned through hole 422 is located is parallel to the abutting plate 6, thereby the drug delivery pipe 41 made of rubber can be easily inserted into the through hole 422. The drug delivery pipe 41 is a silicone hose formed by injection molding. The silicone hose is in the S-shape and communicates the drug delivery hole at the abutting plate and the through hole 422. When the transmission assembly 3 pushes the drug delivery assembly 4 to move, the silicone hose also moves with the drug delivery assembly 4. Since the through hole 422 is parallel to the abutting plate 6, the overall length direction of the through hole 422 is consistent with the movement direction of the silicone hose. In this way, the silicone hose does not need to be deformed too much or the deformation is more balanced during movement, thereby improving the drug delivery efficiency of the drug delivery assembly.

In an embodiment, the filter cavity 21 is provided between the abutting plate 6 and the drug delivery cavity 42. The through hole 422 is closer to the center of the shell 1 compared with the drug delivery hole.

As shown in FIG. 1 and FIG. 2, the abutting plate 6 is attached to the filter cavity 21, the drug delivery cavity 42 is provided on the right of the filter cavity 21, and the drug delivery hole is provided above the abutting plate 6. In this way, the through hole 422 is provided below the abutting hole, that is, the through hole 422 is provided closer to the transmission assembly 3 and is also closer to the center of the shell 1. Therefore, the movement direction of the drug in the drug delivery pipe 41 is from top to bottom, as shown in FIG. 1. That is, the movement direction of the drug in the drug delivery pipe 41 is from a position close to the shell wall to a position away from the center of the shell wall. The center refers to the middle position of the plane of the shell parallel to the connection surface 12. In this way, the cross section of the shell 1 can be gradually reduced from left to right, and the acreage of the cross section of the shell 1 is gradually close to the acreage of the treatment surface 11 on the right.

In an embodiment, a gap is continuously formed between the filter cavity 21 and the drug delivery cavity 42 during the movement of the at least one hollow needle 51 driven by the transmission assembly 3.

In this embodiment, the transmission assembly 3 drives the hollow needle 51 to move, and the hollow needle 51 is fixed in the drug delivery cavity 42, that is, the transmission assembly 3 indirectly drives the drug delivery cavity 42 to move. The filter cavity 21 is provided on the side of the drug delivery cavity 42 close to the abutting plate 6. During the movement of the drug delivery cavity 42, a certain gap between the filter cavity 21 and the drug delivery cavity 42 is always maintained, so that the drug delivery pipe 41 has a certain installation space and/or expansion space.

In an embodiment, the needle board assembly 5 further includes a needle board 52, and the needle board 52 is fixed on the side of the drug delivery assembly 4 away from the transmission assembly 3.

In this embodiment, the needle board assembly 5 further includes a needle board 52, and a hollow needle 51 is fixed on one side of the drug delivery cavity 42. The side of the drug delivery cavity 42 further includes the needle board 52. That is to say, the hollow needle 51 first passes through the needle board 52 and then is inserted into the drug delivery cavity 42. The transmission assembly 3 acts on the side of the drug delivery cavity 42 away from the hollow needle 51 and the needle board 52, and pushes the drug delivery cavity 42, the needle board 52, and the hollow needle 51 toward the treatment area.

In an embodiment, the needle board assembly 5 further includes at least one radio frequency needle 53. Ends of the at least one radio frequency needle 53 are fixed on the needle board 52, and opposite ends of the at least one radio frequency needle 53 are facing with the treatment surface 11.

In this embodiment, the needle board assembly 5 further includes a radio frequency needle 53, which can emit radio frequency energy to act on the treatment area, and can work together with the hollow needle 51 to achieve the treatment effect. One end of the radio frequency needle 53 is fixed on the needle board 52, but is not inserted into the drug delivery cavity 42. The end of the radio frequency needle 53 and the end of the hollow needle 51 facing the treatment surface 11 are in the same horizontal plane, so that when the transmission structure pushes the drug delivery cavity 42, the radio frequency needle 53 and the hollow needle 51 simultaneously penetrate the treatment area.

In an embodiment, the drug includes beauty substances that are beneficial to the skin, such as hyaluronic acid, beauty nutrient solution, and the like. The radio frequency needle 53 and the hollow needle 51 are provided at the needle board assembly 5. The radio frequency needle 53 and the hollow needle 51 are inserted into the treatment area synchronously, and the drug in the hollow needle 51 is introduced into the deep tissue of the skin to accelerate the absorption of the drug by the skin cells. When the radio frequency needle 53 is working, the radio frequency energy is accurately transmitted to the deep tissue of the skin, which will cause the skin to generate heat inside the tissue, thereby selectively heating the target tissue. This thermal energy stimulation can promote the contraction and regeneration of collagen, thereby making the skin tighter and more elastic. The hollow needle 51 can introduce the drug some time after the radio frequency needle 53 releases the radio frequency energy and the target tissue completes the treatment. The drug can help cool the target tissue and relieve pain of the person. In addition, when the drug cools the target tissue, the temperature of the drug will also rise slightly, and the activeness of internal molecules will be higher, which can make the tissue cells absorb the drug more efficiently and quickly. If the drug is hyaluronic acid, the hyaluronic acid is introduced into the skin through the hollow needle 51, thereby providing water-locking function for the skin at a deeper layer, increasing the water molecule content in the skin, and achieving a better water replenishment effect at a deeper layer. If the drug is drug skin drug, nutrients in the drug skin needle can be transported to the inside of the skin, more efficiently fading various blemishes, such as spots, acne marks, pores and fine lines. Therefore, by performing radio frequency energy treatment through the radio frequency needle 53 and introducing the drug through the hollow needle 51 for treatment, the drug can be better absorbed by the treatment area, and the burning pain caused by the radio frequency energy treatment can be reduced.

In an embodiment, the needle board assembly 5 further includes an adapter board 54 and an electrical connector 55. The adapter board 54 is fixed on the abutting plate 6. One end of the electrical connector 55 is electrically connected to the adapter board 54, and an opposite end of the electrical connector 55 is electrically connected to the at least one radio frequency needle 53.

In this embodiment, the radio frequency needle 53 needs to be electrically connected to the treatment handle to release radio frequency energy. The needle board assembly 5 further includes an adapter board 54 and an electrical connector 55. The adapter board 54 is fixed to the abutting plate 6 and is electrically connected to the control board of the treatment handle. The electrical piece 55 electrically connects the adapter board 54 to the radio frequency needle 53, so that the treatment handle can control the radio frequency needle 53 to release radio frequency energy. The adapter board 54 is provided with multiple metal contact points, each metal contact point is connected to a radio frequency needle 53 through an electrical connector 55. The control board of the treatment handle is provided with metal springs which are in the one-to-one correspondence with multiple metal contact points. When the treatment handle is abutted against the treatment tip, the multiple metal springs are respectively abutted against the corresponding metal contact points, so that the control board is electrically connected to the radio frequency needle 53, which facilitates sending the controlled electrical signal to the radio frequency needle 53 through the adapter board 54 and making the radio frequency needle 53 release radio frequency energy.

In an embodiment, there are multiple radio frequency needles 53, and the electrical connector 55 is a flexible circuit board.

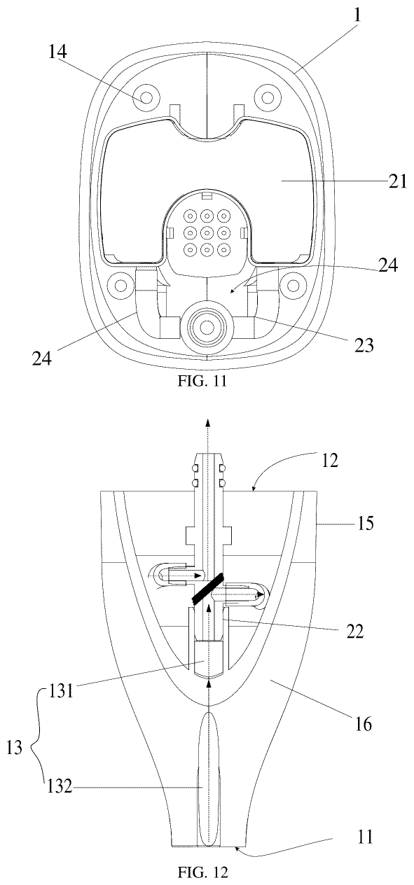

In this embodiment, the electrical connector 55 is a flexible circuit board, and there are multiple radio frequency needles 53. One end of the flexible circuit board is attached to the needle board 52 and is electrically connected to the radio frequency needle 53. In addition, this end of the flexible circuit board is further provided with an avoidance hole corresponding to the hollow needle 51, the avoidance hole can be used for the hollow needle 51 to pass through without affecting the function of the flexible circuit board. In some embodiments, as shown in FIG. 11, the negative pressure assembly 2 is in an annular shape 24. That is, the first pipe, the first inner concave portion 211 of the filter cavity 21, and the second pipe form an annular shape 24. The transmission assembly 3 passes through the annular shape 24 and is attached to the first inner concave portion 211. The flexible circuit board mentioned above also passes through the annular shape 24. One end of the flexible circuit board is fixed on the needle board 52, and an opposite end of the flexible circuit board passes through the abutting plate 6 after passing through the annular shape 24, and is electrically connected to the adapter board 54 of the abutting plate 6. The folding part of the flexible circuit board is provided in the annular shape 24, so that the folding space falls within the annular shape 24 and does not affect other parts when it is pushed by the transmission assembly 3.

In this embodiment, the transmission assembly 3 further includes a transmission hollow pillar 31, which is fixed on the abutting plate 6, and the transmission hollow pillar 31 passes through the annular shape 24 after the transmission hollow pillar 31 passes through the transmission hole.

In this embodiment, the transmission hollow pillar 31 is fixed on the abutting plate 6, and can be integrally formed with the abutting plate 6 by injection molding. The transmission hollow pillar 31 is configured to allow the transmission assembly 3 to pass through. The transmission hollow pillar 31 passes through the transmission hole and then passes through the annular shape 24 formed by the negative pressure assembly 2. The transmission hollow pillar 31 is attached to the portion corresponding to the first inner concave portion 211 of the filter cavity 21, or the transmission hollow pillar 31 has an outer contour that is very like the shape of the inner portion of the first inner concave portion 211, and there is a small gap between the outer contour of the transmission hollow pillar 31 and the the inner portion of the first inner concave portion 211 after installation, which can save space inside the shell 1. In some embodiments, the adapter board 54 has an avoidance hole corresponding to the transmission hole, so that the transmission hollow pillar 31 can passes through the abutting plate and be provided at the abutting plate.

In an embodiment, the transmission assembly 3 includes a push pole 33 and an elastic member 32. An outer boss extends from the end of the push pole 33 away from the treatment surface 11, and an opposite end of the push pole 33 is fixed to the needle board assembly 5. An inner boss is provided inside the transmission hollow pillar 31. The push pole 33 is sleeved inside the transmission hollow pillar 31, and the elastic member 32 is provided between the inner boss and the outer boss.

In an embodiment, the transmission assembly 3 further includes a push board 34, which is fixed between the push pole 33 and the drug delivery cavity 42. The push board 34 is in a planar shape, so that the thrust on the push board 34 is transmitted to the drug delivery cavity 42 through the push board 34, increasing the force-bearing area of the drug delivery cavity 42, and making the drug delivery cavity 42 evenly stressed.

In this embodiment, the push pole 33 is fixed to the power assembly of the treatment handle. The push pole 33 moves toward the treatment surface 11 due to the movement of the power assembly. The cross section of the push pole 33 is in the T-shape. That is, the push pole 33 is provided with an outer boss at one end of the push pole 33 close to the power assembly, and an opposite end of the push pole 33 is fixed to the drug delivery cavity 42. An inner boss is provided at the transmission hollow pillar 31, and an elastic member 32 is provided between the inner boss and the outer boss of the transmission hollow pillar 31. When the power assembly moves, the push pole 33 moves downward, then the inner boss moves downward to squeeze the elastic member 32, and the elastic member 32 is compressed between the inner boss and the outer boss. In this case, the needle board 52 moves downward. Correspondingly, the radio frequency needle 53 and the hollow needle 51 are inserted into the treatment area. When the power assembly withdraws, the external force of the push pole 33 disappears, the elastic member 32 returns to its original state, and the inner boss pushes upward, so that the needle board 52 returns to the initial position, then the radio frequency needle 53 and hollow needle 51 withdraw from the treatment area.

In some embodiments, the push board 34 is fixed to the drug delivery cavity cover 421, or the push board 34 and the above drug delivery cavity cover 421 are an integral structure or the same part.

In an embodiment, the elastic member 32 is a spring, and a spring is sleeved outside the push pole 33. When the spring is sleeved outside the periphery of the push pole 33, the inner boss can be better supported when the spring is restored, then the radio frequency needle 53 and the hollow needle 51 can withdraw from the treatment area.

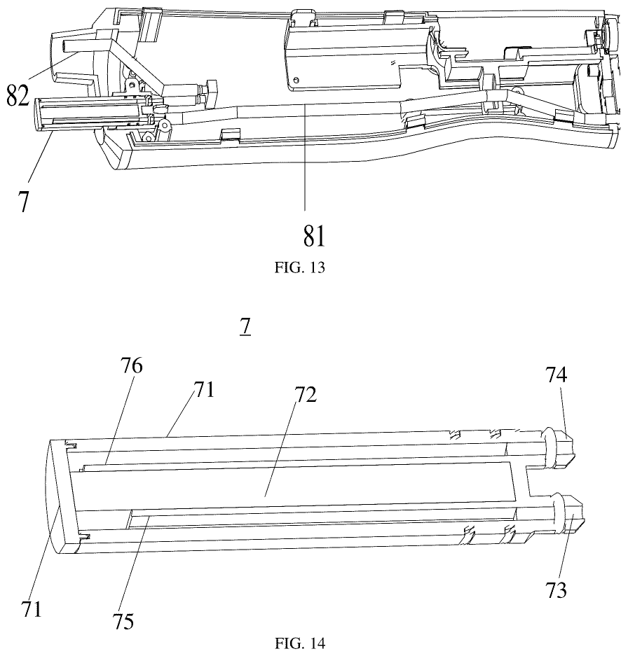

As shown in FIG. 13 and FIG. 14, the present application further proposes a treatment handle, which is detachably connected to the treatment tip according to any embodiment as mentioned above. The treatment handle is provided with an vacuum pump assembly, a power assembly, and a drug push assembly. The vacuum pump assembly communicates with the negative pressure assembly 2. The power assembly is connected to the transmission assembly 3, and the liquid pump communicates with the drug delivery assembly 4. The treatment handle is further provided with a press-type snap fit, which is configured to be fixed with the shell 1, thereby facilitating replacing the treatment tip.

In an embodiment, the vacuum pump assembly includes a first vacuum pump pipe 81 and a second vacuum pump pipe 82. The treatment handle further includes a filter assembly 7. The filter assembly 7 includes a filter shell 71 and a third filter 72. The third filter 72 is provided in the filter shell 71. The filter shell 71 is provided with a second filter inlet 73 and a second filter outlet 74. One end of the first vacuum pump pipe 81 communicates with the negative pressure assembly, and an opposite end of the first vacuum pump pipe 81 communicates with the second filter inlet 73. One end of the second vacuum pump pipe 82 is connected to the second filter outlet 74, and an opposite end of the second vacuum pump pipe 82 is connected to the vacuum pump.

In this embodiment, an vacuum pump configured to generate negative pressure is provided at the main machine. The vacuum pump generates negative pressure, and air with the negative pressure flows along the first vacuum pump pipe 81, the third filter 72, the second vacuum pump pipe 82, and the negative pressure assembly, and then the negative pressure assembly on the treatment tip adsorbs the treatment area. A filter assembly 7 is provided between the first vacuum pump pipe 81 and the second vacuum pump pipe 82 of the treatment handle, to further adsorb the liquid and impurities in at the treatment area.

In an embodiment, the portion of the filter shell 71 opposite to the third filter 72 is composed of the transparent material. This portion of the filter shell 71 protrudes from the treatment handle, or the portion of the outer shell of the treatment handle corresponding to the filter shell 71 is composed of the transparent material, so that the operator can directly see the third filter 72 to get to know the adsorption of the third filter 72, thereby replacing the third filter 72 in time. The third filter 72 can be the sponge.

In an embodiment, a first position-limiting rib 75 and a second position-limiting rib 76 are provided in the filter shell 71. The third filter 72 is provided between the first position-limiting rib 75 and the second position-limiting rib 76. The first position-limiting rib 75 is close to the second filter inlet 73, and the second position-limiting rib 76 is close to the second filter outlet 74. The length of the second position-limiting rib 76 is greater than the length of the first position-limiting rib 75. Two position-limiting ribs with a height difference are provided inside the filter shell 71, which can achieve isolation between water and vapor, thereby more conveniently storing liquid and impurities, and preventing liquid and impurities from entering the vacuum pump.

The present application further proposes a treatment device, which includes a main machine, the treatment tip according to any embodiment as mentioned above, and the treatment handle according to any embodiment as mentioned above. The treatment tip is detachably connected to the treatment handle. The main machine can be fixed to the treatment handle by the cable. The host is provided with an energy source device that provides working energy for the radio frequency needle 53, a vacuum pump that provides negative pressure and other parts, so that the needle board assembly 5, the negative pressure assembly 2 and other related parts on the treatment tip can work normally.

The above are only some embodiments of the present application, and do not limit the scope of the present application thereto. Under the concept of this application, any equivalent structural transformation made according to the description and drawings of the present application, or direct/indirect applied in other related technical fields shall fall within the claimed scope of the present application.

What is claimed is:

1. A treatment tip, comprising:
a shell, provided with a treatment surface configured to contact a treatment area; and
a negative pressure assembly, a transmission assembly, a drug delivery assembly and a needle board assembly; wherein the negative pressure assembly, the transmission assembly, the drug delivery assembly and the needle board assembly are provided inside the shell;
an outlet of the negative pressure assembly is opened on the treatment surface and is configured to apply negative pressure to the treatment area;
an end of the needle board assembly is communicated with an outlet of the drug delivery assembly, and at least one hollow needle is fixed on an opposite end of the needle board assembly;
the at least one hollow needle passes through the treatment surface while the at least one hollow needle is driven by the transmission assembly; and
an end of the drug delivery assembly is communicated with the needle board assembly and is configured to inject the drug into the treatment area through the at least one hollow needle.

2. The treatment tip according to claim 1, wherein:
an abutting plate is connected to an end of the shell facing the treatment surface, and the abutting plate is provided with a negative pressure hole, a transmission hole and a drug delivery hole;
the negative pressure hole is capable for the negative pressure assembly to pass through the abutting plate, the transmission hole is capable for the transmission assembly to pass through the abutting plate, and the drug delivery hole is capable for the drug delivery assembly to pass through the abutting plate; and
the negative pressure assembly comprises a filter cavity, and the filter cavity is fixed to a surface of the abutting plate of which the surface is close to the treatment surface.

3. The treatment tip according to claim 2, wherein the filter cavity comprises a first inner concave portion; the transmission hole is provided inside a projection of the first inner concave portion onto the abutting plate; and
the filter cavity is provided with a first filter inlet and a first filter outlet; the negative pressure assembly further comprises a first negative pressure pipe and a second negative pressure pipe; the first negative pressure pipe is connected between the first filter inlet and the treatment surface; and
the second negative pressure pipe is connected between the first filter outlet and the negative pressure hole.

4. The treatment tip according to claim 3, wherein:
a negative pressure hollow pipe is integrally formed at a shell wall of the shell facing the first negative pressure pipe; and
an end of the negative pressure hollow pipe is communicated with the treatment surface, and an opposite end of the negative pressure hollow pipe is communicated with the first negative pressure pipe.

5. The treatment tip according to claim 4, wherein:
the shell comprises a first portion and a second portion; the first portion is provided with the abutting plate, and the second portion is provided with the treatment surface;
an outer contour of the second portion gradually shrinks from a portion of the second portion contacting with the first portion toward the treatment surface;
the negative pressure hollow pipe comprises an inner hollow pipe and an outer hollow pipe communicated with the inner hollow pipe;
the inner hollow pipe is protruded inwardly from an inner wall of the first portion and/or an inner wall of the second portion; and
the outer hollow pipe is protruded outwardly from an outer wall of the second portion.

6. The treatment tip according to claim 3, wherein the first negative pressure pipe, the second negative pressure pipe and the first inner concave portion are communicated as a negative pressure loop which is projected on the abutting plate as an annular ring, and the transmission hole is disposed inside the annular ring.

7. The treatment tip according to claim 3, wherein a distance between the first filter outlet and the abutting plate is smaller than a distance between the first filter inlet and the abutting plate.

8. The treatment tip according to claim 2, wherein:
the filter cavity further comprises a second inner concave portion;
the drug delivery assembly comprises a drug delivery pipe, and the drug delivery pipe passes through the second inner concave portion after passing through the drug delivery hole;
the drug delivery assembly is provided with a drug delivery cavity;
ends of the at least one hollow needles are fixed with the drug delivery cavity, and opposite ends of the at least one hollow needle are facing with the treatment surface; and
the drug delivery cavity is connected to the drug delivery pipe.

9. The treatment tip according to claim 8, wherein the drug delivery pipe is a silicone hose that is formed integrally; the drug delivery cavity comprises a through hole communicated with the drug delivery pipe, and a plane where the through hole is disposed is perpendicular to the abutting plate.

10. The treatment tip according to claim 8, wherein the drug delivery pipe is a silicone hose that is formed integrally; the drug delivery cavity comprises a through hole communicated with the drug delivery pipe, and a plane on which the through hole is disposed is parallel to the abutting plate.

11. The treatment tip according to claim 9, wherein:
the filter cavity is disposed between the abutting plate and the drug delivery cavity;
the through hole is disposed closer to a center of the shell compared with the drug delivery hole; and
a gap is continuously formed between the filter cavity and the drug delivery cavity during the movement of the at least one hollow needle driven by the transmission assembly.

12. The treatment tip according to claim 2, wherein the needle board assembly further comprises a needle board, and the needle board is fixed on a side of the drug delivery assembly away from the transmission assembly; the needle board assembly further comprises at least one radio frequency needle; ends of the at least one radio frequency needle are fixed on the needle board, and opposite ends of the at least one radio frequency needle are facing with the treatment surface.

13. The treatment tip according to claim 12, wherein the needle board assembly further comprises an adapter plate and an electrical connector; the adapter plate is fixed on the abutting plate; an end of the electrical connector is electrically connected to the adapter plate; an opposite end of the electrical connector is electrically connected to the at least one radio frequency needle.

14. The treatment tip according to claim 6, wherein the transmission assembly comprises a transmission hollow pillar; the transmission hollow pillar is fixed on the abutting plate, and the transmission hollow pillar passes through the annular shape after the transmission hollow pillar passes through the transmission hole; and
the transmission hollow pillar is attached to a portion of the filter cavity corresponding to the first inner concave portion of the filter cavity.

15. The treatment tip according to claim 14, wherein:
the transmission assembly comprises a push pole and an elastic member; the push pole has an outer boss extending from an end of the push pole away from the treatment surface, and an opposite end of the push pole is fixed to the needle board assembly; and
an inner boss is provided inside the transmission hollow pillar; the push pole is sleeved inside the transmission hollow pillar, and the elastic member is provided between the inner boss and the outer boss.

16. A treatment handle, detachably connected to the treatment tip according to claim 1, comprising:
a vacuum pump assembly;
a power assembly; and
a drug push assembly;
wherein the vacuum pump assembly is communicated with the negative pressure assembly; the power assembly is connected to the transmission assembly, and the drug push assembly is communicated with the drug delivery assembly.

17. The treatment handle according to claim 16, wherein a press-type snap fit is provided at the treatment handle, and the press-type snap fit is clamped with the shell.

18. The treatment handle according to claim 16, wherein:
the vacuum pump assembly comprises a first vacuum pump pipe and a second vacuum pump pipe, and the treatment handle further comprises a filter assembly;
the filter assembly comprises a filter shell and a third filter; the third filter is provided inside the filter shell, and the filter shell is provided with a second filter inlet and a second filter outlet;
an end of the first vacuum pump pipe is communicated with the negative pressure assembly, and an opposite end of the first vacuum pump pipe is communicated with the second filter inlet;
an end of the second vacuum pump pipe is connected to the second filter outlet, and an opposite end of the second vacuum pump pipe is connected to an vacuum pump.

19. The treatment handle according to claim 18, wherein:
a first position-limiting rib and a second position-limiting rib are provided inside the filter shell, and the third filter is disposed between the first position-limiting rib and the second position-limiting rib;
the first position-limiting rib is disposed close to the second filter inlet, and the second position-limiting rib is close to the second filter outlet; and
a length of the second position-limiting rib is greater than a length of the first position-limiting rib.

20. A treatment device, comprising:
a host;
the treatment tip according to claim 1; and
a treatment handle comprising:
a vacuum pump assembly;
a power assembly; and
a drug push assembly;
wherein the vacuum pump assembly is communicated with the negative pressure assembly;
the power assembly is connected to the transmission assembly, and the drug push assembly is communicated with the drug delivery assembly;
the treatment tip is detachably connected to the treatment handle; and
the host is connected to the treatment handle.
